(19) United States
(12) Patent Application Publication
(10) Pub. No.: US 2014/0296662 A1
(43) Pub. Date: Oct. 2, 2014

(54) METHOD AND DEVICE FOR DETECTING THE STATE OF HYDRATION OF A HUMAN OR ANIMAL BODY

(71) Applicant: Robert Bosch GmbH, Stuttgart (DE)

(72) Inventors: Henning Hayn, Stuttgart (DE); Kathrin Graner, Fellbach (DE)

(73) Assignee: Robert Bosch GmbH, Stuttgart (DE)

(21) Appl. No.: 14/227,797

(22) Filed: Mar. 27, 2014

(30) Foreign Application Priority Data
Mar. 27, 2013 (DE) 10 2013 205 403.3

(51) Int. Cl. A61B 5/00; A61B 5/0402; A61B 5/0205

(52) U.S. Cl. CPC A61B 5/4875; A61B 5/443; A61B 5/0205; A61B 5/0402; A61B 5/7278; A61B 5/0006; A61B 5/6825; A61B 5/6829; A61B 5/6833; A61B 5/706; A61B 5/0537
USPC 600/306; 600/547; 600/483; 600/384

(57) ABSTRACT

A method for detecting the state of hydration of a human or animal body includes carrying out impedance measurements on a body surface of the human or animal body. At least one bipole measurement and at least one quadrupole measurement are carried out and evaluated during the method.

This application claims priority under 35 U.S.C. §119 to patent application no. DE 10 2013 205 403.3 filed on Mar. 27, 2013 in Germany, the disclosure of which is incorporated herein by reference in its entirety.

### BACKGROUND

The present disclosure relates to a method and a device for detecting the state of hydration of a human or animal body.

The state of hydration of a person is usually described as the level of the proportion of fluid in the human body. If the body contains too little fluid, there is the danger of drying out. Dehydration is spoken of in this case. Depending on how the ratio between water content and salt content in the body changes, dehydration is spoken of as being isotone (loss of water and salt in the same ratio), hypertone (water loss without corresponding salt loss) or hypotone (salt loss higher than the water loss). By contrast, hyperhydration is spoken of if too much water is present in the body. It is chiefly older people who frequently suffer from dehydration which is accompanied by serious consequences for health. It is therefore necessary to be able to monitor a person's state of hydration as regularly and simply as possible in order to recognize the risk of dehydration in good time so that suitable measures, in particular a supply of fluid, can be taken.

There are already various methods which enable a person's state of hydration to be determined. For example, it is possible to measure the specific urine gravity, or the state of hydration can be determined with the aid of blood values, or a Turgor test can be carried out. A further possibility is to estimate the state of hydration with the aid of the coloration of the mucous membrane of the mouth. It is also possible to infer the state of hydration by considering the elasticity of the skin, although the skin elasticity is reduced in particular in older people, from which it follows, above all for the risk group of older people, that a clear statement on the state of hydration can be made only with difficulty.

Furthermore, the state of hydration of a body can be determined by means of a bioelectric impedance measurement. Bioelectric impedance measurement utilizes the fact that the total impedance of the human body is determined from the different conductivities of the various body components. This method can therefore be used to investigate the body composition, it being possible, in particular, to determine the total body water content and the muscle mass/fat mass.

German Laid-Open Patent Application DE 10 2006 002 142 A1 describes a medical measuring instrument with an impedance measuring unit, a resistance value and a reactance value being determined from the impedance measurement signal, and the body cell mass and the state of hydration of the respective person being inferred from said values. Here, the resistance describes the ohmic proportion of the measurement, which is determined substantially by the conductivity of the extracellular fluid. The reactance describes the capacitive proportion of the measurement, which is determined substantially by the conductivity of the cell membranes and the intracellular fluid.

### SUMMARY

The method for detecting the state of hydration of a human or else animal body is based on impedance measurements on the body surface. According to the disclosure, in this case at least one bipole measurement and at least one quadrupole measurement are carried out and evaluated. The core of the disclosure is switching over between a bipole and a quadrupole measuring arrangement. Different parameters of the body composition can be determined with respect to the state of hydration of the body by combining these two measurement principles. Consequently, an evaluation of the measurement results permits very accurate and complex statements relating to the state of hydration of the patient, and this without the need to provide a high outlay on measurement and within a short measuring time. Consequently, the method is suitable for an easy and uncomplicated detection of the state of hydration such that, for example, it is possible straight away to monitor a patient regularly without impairing the quality of life. Reliable statements relating to the state of hydration can be made in a very short time owing to the combination of the bipole and the quadrupole measurement principles and a corresponding evaluation of the measurement results. By way of example, there is no need for a blood sample or for a urine test. The method is very user-friendly and quick and can therefore, for example, be used straight away in a care institution or at home. In comparison with conventional bioimpedance measurements, it is also possible, using the method, to make substantially more accurate statements relating to the state of hydration owing to the combination of bipole and quadrupole measurements.

For the bipole measurement, two electrodes are attached to, or used at, the measurement locations, for example to/at the hands and/or to/at the feet. The measurement current is coupled in via said electrodes. Furthermore, the dropping voltage is also tapped via said electrodes. In this process, the electrode impedances, the skin impedance and the tissue impedance enter into the voltage drop. That is to say, the contact impedances of the electrodes and the skin impedance are also measured in addition to the tissue impedance in the case of bipole measurement. In the case of quadrupole measurement, four electrodes are attached to, or used at, the measurement locations. The current is coupled in via the two outer electrodes. The dropping voltage is tapped via the two inner electrodes. It is approximately the case here that the measured impedance corresponds to the tissue impedance, this being so on the assumption that the input impedance of the measuring unit is greater than the tissue impedance, the electrode impedances and the skin impedance. Since this is the case as a rule for measuring unit currently customary, this means that the electrode impedances and the skin impedance can be neglected, and so the measured impedance corresponds approximately to the tissue impedance.

The method enables a detailed analysis of the water content in the body owing to the combined measurement of the body impedance via a bipole and a quadrupole circuit. In particular, the tissue impedance is measured with the aid of the quadrupole measurement. The skin impedance can be inferred from the valves of the bipole measurement taking account of the measured tissue impedance. Thus, the skin impedance can be detected by taking into account the measurement results for the two measurement arrangements. Thus, very informative and detailed statements relating to the water content in the body can be made from the combination of the different measurement principles and a corresponding evaluation.

The measurement of the impedances is performed by applying an alternating current, preferably with a constant current. The measurements are expediently carried out with different frequencies. Depending on the measurement frequency, the total body water content, the intracellular body water content and the extracellular body water content can be inferred from the measurement results of the quadrupole measurements. Thus, together with the skin impedance, which can be detected by the bipole measurement, it is easily possible to measure four parameters which are all correlated with the state of hydration of a person. By comparison with conventional measurement methods, the state of hydration can be determined substantially more exactly by an evaluation of said parameters such that, for example, it is possible to determine the risk of dehydration of a patient very accurately from the state of hydration. In turn, this permits real time intervention, for example by the care staff, so that it is possible to avoid health restrictions owing to dehydration, and thus also to save costs.

The inferences from the measured impedances to the total body water content, the skin water content, the intracellular body water content and/or the extracellular body water content can be made by applying algorithms, in particular data concerning the body size and/or concerning the body weight and/or concerning the sex and/or concerning the age of the patient or user being input into the algorithms. Simple formulas can form the basis for suitable algorithms. The basis for this can be supplied, for example, by empirical values, it being possible to take into account that with increasing age the water content in the body decreases and/or that the body water proportion differs as a rule between women and men. In addition to the impedance values, the phase angle of the measurement results can also be taken into account for an evaluation, the phase angle describing the ratio of resistance to reactance. It is known that the phase angle is a general dimension for the state of nutrition at the cell level, and so this information can also be input for evaluating the state of hydration which is detected in accordance with the disclosure.

When the measurements are being carried out, it is advantageous to undertake a preliminary measurement of the electrode impedances, said measurement results being taken into account in the evaluation. This is advantageous, in particular, for the evaluation of the bipole measurements, since the skin impedance can be inferred directly from the measurement results of the bipole measurement by taking account of the electrode impedance, which can be assumed to be constant, and given that account is taken of the tissue impedance, which can be detected via the quadrupole measurement.

It is advantageous for a plurality of measurements to be carried out to form mean values. For example, bipole measurements can be carried out in which two electrodes are respectively used on an extremity. From measurements at each extremity, that is to say four measurements, it is possible to form a mean value of the skin impedance which enables a very reliable statement concerning the skin impedance, and thus concerning the state of hydration.

In a further refinement of the method, additional measurements can be carried out to derive an electrocardiogram, it being possible to use the electrodes which are provided in accordance with the disclosure for the impedance measurements in order to detect the state of hydration. In principle, two electrodes are sufficient for this purpose, more electrode derivations also being possible, however. A precondition for deriving an electrocardiogram is that the current path is guided via the heart.

In a particularly advantageous refinement of the method, the detection of the state of hydration is incorporated into a telemedical system. Since the method can be carried out very easily and quickly and with little outlay, it is particularly suitable for application in a care home or at home, the patient himself being able to carry out the measurement straight away. The results relating to the detected state of hydration can then be passed on via suitable interfaces, for example a USB interface, Bluetooth, WLAN or similar, so that they can be evaluated by specialists at another location, for example in a practice or a hospital. Any required measures can then be taken. The required technical preconditions, that is to say suitable interfaces, in particular, can be integrated in the respective measuring unit.

The disclosure further comprises a device for detecting the state of hydration of a human or else animal body which is based on the measurement of impedances. Provided in this device are at least four, in particular at least eight, electrodes which are to be attached to the body surface, in particular to the hand surfaces and/or the soles of the feet. Further provided is a current source which is used to apply alternating current to the electrodes. Essential to the inventive device is the fact that the electrodes can be switched both for bipole measurements and for quadrupole measurements. The inventive device is suitable for carrying out the above-described method, it being possible to make detailed statements relating to the state of hydration of a patient by a combination of bipole measurements and quadrupole measurements and an appropriate evaluation of the measurement results. In particular, said device can be used to measure the total body water content, the intracellular water content, the extracellular water content and the state of hydration of the skin. The current source of the inventive device is configured such that alternating current can be applied to the electrodes. It is preferably possible to set various measurement frequencies. Different aspects of the state of hydration can be investigated by measurements with different frequencies. In particular, it is possible by means of different measurement frequencies to distinguish between the total body water content, the intracellular water content and the extracellular water content.

It is advantageous to use electrodes with a relatively low contact impedance for the inventive device and the method. Suitable examples are electrodes with a relatively large surface and/or the use of materials which are good conductors such as, for example, silver chloride and/or ruthenium black. It is thereby possible for the contact impedances of the electrodes to be minimized, particularly with regard to the bipole measurement, so that the measurement resolution is increased.

Furthermore, the device can comprise a control unit, a suitable evaluation unit, that is to say, in particular, suitable means for data processing, and suitable operating units and display units, for example a display for showing the measurement results and/or the evaluation of the measurement results.

The inventive device can expediently have a patient protection circuit which prevents excessively high currents which can be harmful in some circumstances from flowing through the patient during measurement.

The electrodes of the device can be, for example, conventional adhesive electrodes which can be fastened to the hand surfaces and/or the soles of the feet of a user. It is also possible, however, to use dry, non-adhesive electrodes. It is particularly advantageous when the electrodes are integrated in handgrips and/or standing surfaces of the measuring unit. This permits a particularly easy manipulation of the device, which can be undertaken straight away by a patient himself. In this refinement, the electrodes have a particularly long service life and are integrated in the measuring device to suit the patient.

The inventive measuring device can be integrated in a telemedical system, interfaces being provided in the device which enable the measured data or the evaluated data to be passed on, for example, to a central computer which can be set up in a practice or a hospital, for example.

Finally, the disclosure comprises a computer program and a computer program product for carrying out the method for detecting the state of hydration and, in particular, for carrying out the impedance measurements and for evaluating the measurement results when the program is executed on an arithmetic unit or a control unit. Such a program can, for example, be integrated in the inventive device. By way of example, it is also possible for the device to carry out solely the measurements. The measurement results are then passed on via an appropriate interface to a computer, for example, in which the measurement results are evaluated.

Further features and advantages of the disclosure emerge from the following description of exemplary embodiments in conjunction with the drawings. The individual features can be implemented in this case on their own, respectively, or in combination with one another.

### BRIEF DESCRIPTION OF THE DRAWINGS

In the drawings:

FIG. 1A shows a diagrammatic illustration of a bipole measurement;

FIG. 1B shows an equivalent circuit diagram of the arrangement of FIG. 1A;

FIG. 2A shows a diagrammatic illustration of a quadrupole measurement;

FIG. 2B shows a corresponding equivalent circuit diagram of the arrangement of FIG. 2A;

FIG. 3 shows an equivalent circuit diagram illustrating the measurement of the total body water content;

FIG. 4 shows a diagrammatic illustration of a measurement setup for detecting the state of hydration of a person;

FIG. 5 shows a circuit diagram for impedance measurements in accordance with the disclosure;

FIG. 6 shows a circuit diagram for switching over between a bipole circuit and a quadrupole circuit by short circuiting two electrodes, and

FIG. 7 shows a circuit diagram for switching over between a bipole circuit and a quadrupole circuit by excluding two electrodes.

### DETAILED DESCRIPTION

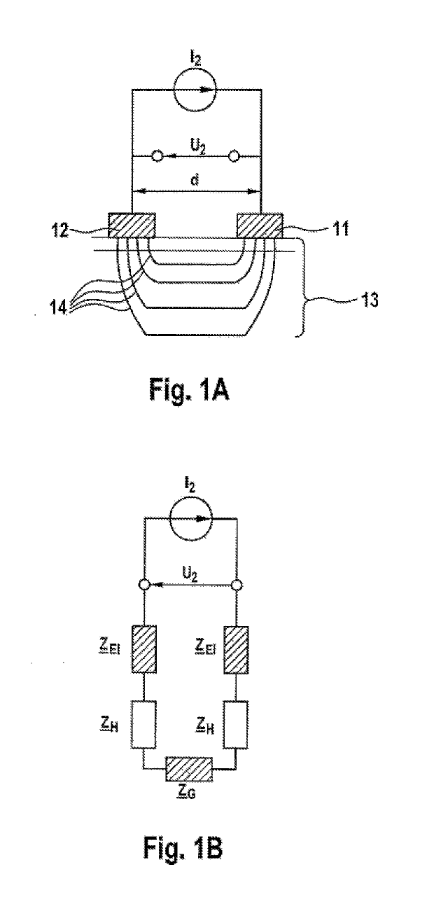

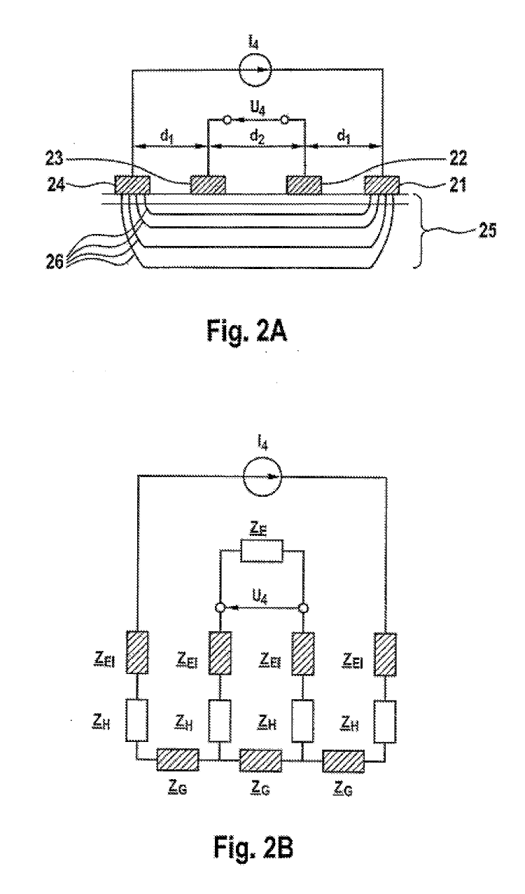

FIGS. 1 and 2 firstly illustrate the principles of a bipole measurement (FIGS. 1A and 1B) and a quadrupole measurement (FIGS. 2A and 2B) by means of which the impedance measurements can be carried out on the body surface of a patient or user in order to detect the state of hydration of the body. According to the disclosure, said two measurement principles are combined with one another and appropriately evaluated in order to be able to make a detailed statement concerning the state of hydration of the patient. In particular, by combining said measurement principles it is possible to determine the total body water content, the intracellular water content, the extracellular water content and the skin water content, it being possible to measure and/or calculate the corresponding impedances and, in particular, to infer said various parameters of the state of hydration from the measured and calculated impedances by applying suitable algorithms.

The principle of bipole measurement is illustrated in FIG. 1A. Here, two electrodes 11 and 12 are attached to the body at the measurement locations (FIG. 1A). The measurement current (I₂) is coupled into the object via the electrodes 11 and 12. Furthermore, the voltage (U₂) dropping at the object is tapped via the electrodes 11 and 12. The skin 13, on which the electrodes 11 and 12 rest, is subdivided diagrammatically into two layers through which the power lines 14 run. FIG. 1B shows an equivalent circuit diagram of said arrangement. The alternating current resistances (impedances Z) for the electrodes are denoted by Z_{EI}. The impedances for the skin are denoted by Z_{H}. The tissue impedance is denoted by Z_{G}. In accordance with Ohm's law, the impedance (Z₂) lying between the two electrodes 11 and 12 can be calculated as follows:

$${\underline{Z}}_{2} = {\underline{Z}}_{G} + 2 {\underline{Z}}_{H} + {\underline{Z}}_{EI} = \frac{{\underline{U}}_{2}}{{\underline{I}}_{2}}$$

Here, all the impedances which lie on the path of the current from one electrode to the other are taken into account. For the bipole measurement, these are thus, in particular, the first electrode impedance (Z_{EI}), the skin impedance (Z_{H}), the tissue impedance (Z_{G}), the skin impedance once again, and, finally, the impedance of the second electrode. Consequently, in addition to the tissue impedance the contact impedances of the electrodes and the skin impedance are also measured in the bipole measurement. For the bipole measurement, it is possible, in particular, to use electrodes with a relatively low contact impedance in the course of the method, for example electrodes which are as large as possible. Alternatively or in addition, it is possible to use particularly well conducting materials such as, for example, silver chloride or ruthenium black in order thus to keep the electrode impedance low and thereby increase the measurement resolution.

FIG. 2A illustrates in comparable fashion a quadrupole measurement in which four electrodes 21, 22, 23 and 24 are attached to the measurement locations. The current (I₄) is coupled into the measurement object via the two outer electrodes 21 and 24. The dropping voltage (U₄) is tapped via the two inner electrodes 22 and 23. The current running through the skin 25 is indicated by the power lines 26. FIG. 2B shows a corresponding equivalent circuit diagram, the various alternating current resistances at the electrodes, the skin and the tissue being correspondingly denoted as impedances as in FIG. 1B. It thus follows from a setup of the current and voltage ratios at the individual impedances to be taken into account that:

$$\frac{{\underline{U}}_{4}}{{\underline{I}}_{4}} = \underline{Z} = \frac{{\underline{Z}}_{G}}{1 + \frac{{\underline{Z}}_{G}}{{\underline{Z}}_{E}} + 2 \frac{{\underline{Z}}_{EI}}{{\underline{Z}}_{E}} + 2 \frac{{\underline{Z}}_{H}}{{\underline{Z}}_{E}}} \approx {\underline{Z}}_{G}$$

Here, the measured impedance corresponds approximately to the tissue impedance, this being so on the assumption that the input impedance Z_{E} of the measuring unit is very much greater than the impedances Z_{G}, Z_{EI} and Z_{H}. This condition is fulfilled as a rule by conventional measuring units. Consequently, the electrode impedances and the skin impedance can be neglected. By contrast with a bipole measurement, the measurement resolution of a quadrupole measurement is therefore generally higher. The method combines the bipole measurement and the quadrupole measurement so that the different resolutions and measurement ranges involved with said measurement principles can be used and input into the detection of the state of hydration.

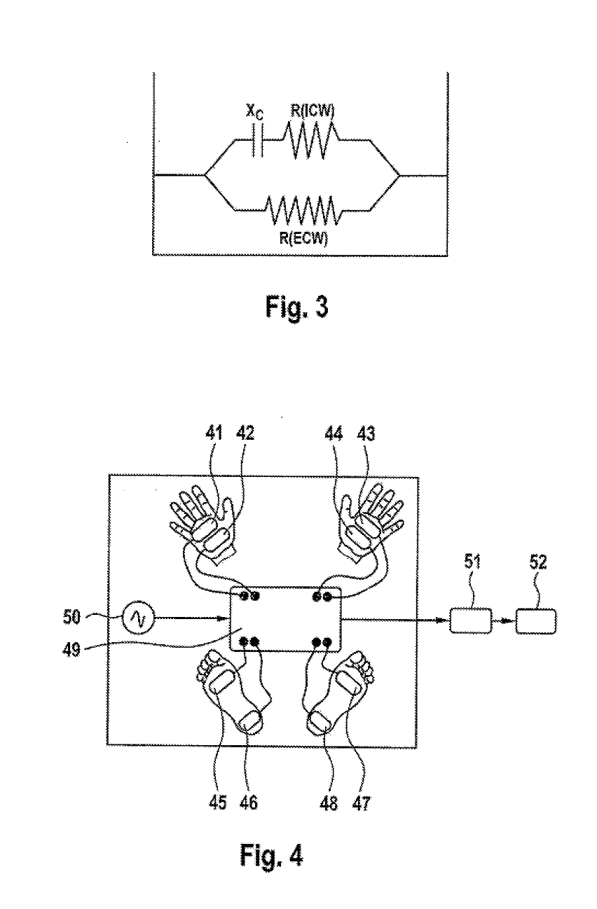

By combining the impedances measured by a bipole arrangement and by a quadrupole arrangement, it is possible to make both statements relating to the tissue impedance and statements relating to the skin impedance. Furthermore, different measurement frequencies render it possible in the case of a quadrupole arrangement to make statements relating to the total body water and to the intercellular water and the extracellular water. Here, FIG. 3 illustrates the basic principle as an equivalent circuit diagram which takes account of the capacitive properties of the cell membranes (X_{C}), and of the resistance caused by the intracellular fluids (R(ICW)) and by the extracellular fluids (R(ECW)). Here, ICW stands for intracellular fluid within the cell membranes, ECW for the extracellular fluid, including the blood, and TBW for the total body fluid. Fluid and water are to be understood as synonymous. The total body water TBW is formed from the sum of the intracellular water ICW and the extracellular water ECW (TBW=ECW+ICW). It follows from the illustrated equivalent circuit diagram that, given a frequency of 0 kHz, the resistance due to the extracellular water R(ECW) acts as dominant resistance, since the capacitive properties of the cell membranes X_{C} tend to infinity. When X_{C} tends to 0, that is to say given a parallel circuit composed of R(ECW) and R(ICW), the total body water and/or the resistance is/are measured. Thus frequencies as high as possible, for example f(TBW)=50 kHz, are used to measure the total body water resistance. Frequencies as low as possible, for example f(ECW)=5 kHz, are used to measure the extracellular water and/or the corresponding resistance. The resistance of the intracellular fluid results from the equation ICW=TBW−ECW, and so it is possible to infer ICW from the measurement results relating to TBW and ECW. Measurements with f(TBW) and f(ECW) are therefore sufficient to be able to make statements relating to all three aspects (TBW, ECW, ICW).

As has already been stated above, both the electrode impedances and the tissue impedances play a role in the determination of the skin impedance by the bipole measurement. Consequently, in order to determine the skin impedance it is advantageous to determine the impedance of the electrodes in advance and regard it as constant. Possible corrections can be undertaken by regular calibration and/or checking of the electrode impedances. In determining the skin impedance, the tissue impedance (Z_{G}) is likewise taken into account, being determined with the quadrupole measurement. The tissue impedance detected in the course of the quadrupole measurement can be used to calculate the skin impedance in the following way:

$${\underline{Z}}_{H} = \frac{1}{2} \cdot ( {\underline{Z}}_{2} - {\underline{Z}}_{G} - 2 {\underline{Z}}_{EL}$$

Thus, all that is required to determine the skin impedance is a simple calculation.

It is advantageous to produce correlations relating to the state of hydration with reference to the particular patient or user from the various impedance values determined in accordance with the disclosure. It is possible to this end to use various algorithms which can be derived, in particular, on the basis of empirical values, the age and other factors which play a role in the body water content, for example, being taken into account.

A suitable algorithm for calculating the body water on the basis of the impedance measurements in accordance with the disclosure is based, for example, on a model which represents a person in a simplified fashion as a cylinder. The electrical resistance of such as electric conductor (in ohms) is:

$$R = ρ \cdot \frac{L}{A}$$

ρ representing the specific, material-dependent resistance, L the conductor length, that is to say, in particular, the body length in centimeters, and A the cross-sectional area of the conductor. Since it is possible to determine the cross-sectional area of a person only with difficulty, this is replaced by the quotient

$$A = \frac{volume}{body length} = \frac{V}{L} ,$$

resulting in the following relationship:

$$R = ρ \cdot \frac{{L}^{2}}{V} .$$

Values for the resistance R and for the alternating current resistance (impedance) are determined by using various measurement frequencies, it being possible for the impedance Z to be described as Z=R+j X, R describing a real part, X an imaginary part and j an imaginary unit. The volume can be equated to the body water. Whether what is involved here is the extracellular, the intracellular or the total body water depends on the measurement frequency respectively used.

Since a person is not a homogeneous body, it is not possible to determine a specific resistance. Consequently, use is made of empirically determined constants (K) which, depending on the target group under consideration, can vary as a function of age and sex, for example. The water quantity in liters yielded by the last-described equation is:

$$Water quantity = {K}_{1} \cdot \frac{{L}^{2}}{R}$$

Since the proportion of water depends, furthermore, on the sex, the age (a; in years) and weight (m; in kilograms), this formula is expanded and corrected with further summands. The result in general is thus:

$$Water quantity = {K}_{1} \cdot \frac{{L}^{2}}{{R}_{f}} + {K}_{2} \cdot m + {K}_{3} \cdot a + {K}_{4} ,$$

use being made of different constants K determined empirically for the sexes and the various age groups.

FIG. 4 illustrates diagrammatically a possible setup for carrying out the method. In this refinement, eight electrodes 41-48 are provided, of which respectively two are attached to the two hand surfaces and the two soles of the feet of a patient. In this case, the electrodes can be adhesive electrodes or other dry, non-adhesive electrodes. Furthermore, the electrodes can also be integrated in handgrips and/or standing surfaces of a measuring unit. According to the disclosure, there is alternation between a bipole measurement and a quadrupole measurement. Consequently, the measuring unit is designed so that in the case of a bipole measurement only the two electrodes required for the measurement are driven, while the other electrodes are deactivated, that is to say no current flows through them. This deactivation is cancelled for the quadrupole measurement. Furthermore, it is advantageous when the bipole measurements and the quadrupole measurements are carried out on different body locations, that is to say thus with different electrodes, for example. It is therefore expediently possible for each electrode to be both deactivated and also switched on. By way of example, it is possible to measure using only two electrodes on the surface of the left hand or, for example, to drive respectively one electrode on each extremity in a quadrupole measurement. The sequence of the driving of the electrodes can be fixed by the measurement protocol. For example, it is possible firstly to carry out quadrupole measurements for determining TBW, ICW and ECW with the aid of suitable measurement frequencies. It is possible to this end, for example, to drive firstly the electrodes of the right-hand side of the body, then the electrodes of the left-hand side of the body and subsequently respectively one electrode on each extremity. Subsequently, a bipole measurement can be carried out to determine the skin impedance. By way of example, to this end, four measurements can be carried out with respectively two electrodes on each extremity such that a mean value of the skin impedance can then be determined.

The individual electrodes are driven via the circuit 49 and by means of the current source 50. The frequencies applied are variable in this case and are set as a function of the parameter respectively to be measured. The measurement results are processed and evaluated using suitable algorithms in a data processing unit 51. The results relating to the individual parameters of the state of hydration, or a summary result can be displayed via the output unit 52. A corresponding result can, for example, be printed out and/or showed on a display. The measured data and/or the evaluated result can also, for example, be passed on and displayed at another location in the course of telemedical applications.

By way of example, the electrodes 41, 43, 45, 47 or 42, 44, 46, 48 or 43, 44, 47, 48 or 41, 42, 45, 46 or 43, 44, 45, 46 or 41, 42, 47, 48 can be used to determine TBW, ICW and EWC, which are measured with the aid of a quadrupole arrangement. By way of example, the electrodes 41, 42 or 43, 44 or 45, 46 or 41, 45 or 47, 48 or 42, 46 or 43, 47 or 44, 48 can be used to measure the skin impedance Z_{H}, which is measured with the aid of a bipole arrangement. If, in addition, the aim is to derive a cardiogram, this can be done, for example, by using the electrodes 41, 43 or 42, 44 or 43, 45 or 41, 47.

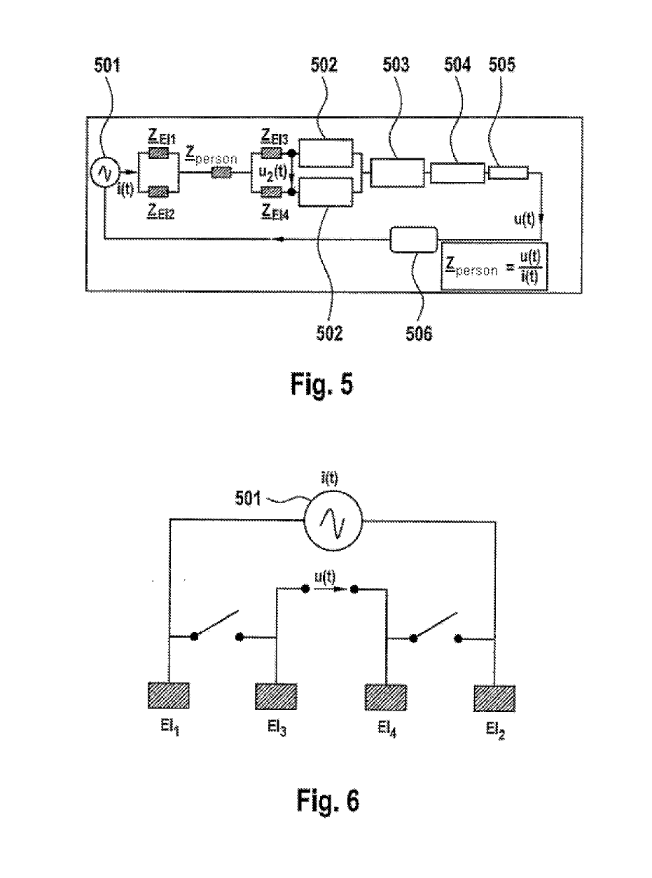

FIG. 5 shows a general circuit diagram for the impedance measurements in the course of the method in order to detect the state of hydration of a body. Variable measurement frequencies are applied to the various electrodes with the aid of a current source 501, in particular an alternating current generator. The measurement sequence is controlled via the control unit 506. Four different electrodes are driven in the case of quadrupole measurements. It holds in this case that: Z_{EI1}≠Z_{EI2}≠Z_{EI3}≠Z_{EI4}. If measurement is done in a bipole arrangement, the voltage is tapped via the electrode pair via which the current is also fed into the body. It is therefore possible to understand the circuit diagram in FIG. 5 in the sense that Z_{EI3}=Z_{EI1} and Z_{EI4}=Z_{EI2}. The measurable impedances are denoted in general as Zₚₑᵣₛₒₙ. Also used in the circuit are an impedance converter 502, a difference amplifier 503, a further amplifier 504 and an A/D converter 505. The results for the impedance measurement, which represent the proportion of the measurable voltage as a function of the impressed alternating current, can be processed and evaluated in the control unit 506, for example a computer, a microcontrol unit or, in general, a control unit. Data processing can also be performed externally.

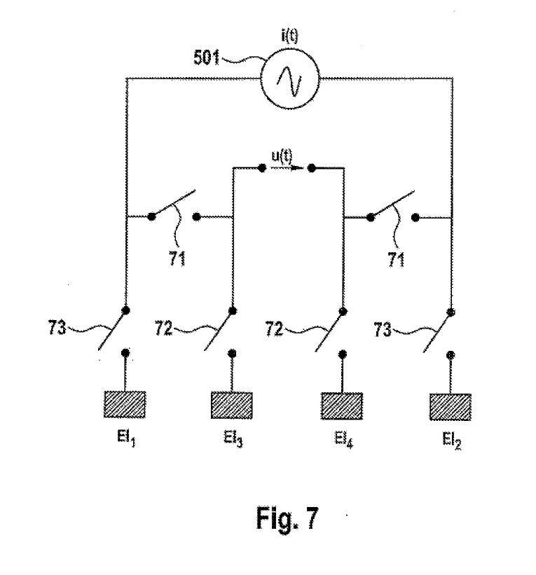

Switching over between bipole measurements and quadrupole measurements is essential to the inventive device. Exemplary circuit diagrams for possible switchovers are illustrated in FIGS. 6 and 7. A switchover by means of a short circuit between respectively two electrodes is illustrated in FIG. 6. If both switches are open, measurement is performed using four electrodes. In the case of closed switches, the electrode EI₁ is short circuited with the electrode EI₃, and the electrode EI₂ is short circuited with the electrode EI₄, resulting in the measurement being performed via two electrodes. However, the latter have twice the surface area of the original measurement electrodes. If there is no desire for such a change in surface area, it is possible, by way of example, to perform measurement with a variant of the switchover, as is illustrated in FIG. 7. In this variant, the electrodes are not short circuited, but two further pairs of switches are used to exclude two electrodes from the measurement circuit. The pair of switches 71 is opened for the quadrupole measurement. The pairs of switches 72 and 73 are closed. For a bipole measurement with the two outer electrodes EI₁ and EI₂, the pair of switches 71 and 73 are closed, while the pairs of switches 72 remains open. The connection to the two inner electrodes EI₃ and EI₄ is thereby interrupted. If, by contrast, measurement is performed using the two inner electrodes EI₃ and EI₄, the pairs of switches 71 and 72 are closed, while the pair of switches 73 remains open. Connection to the electrodes EI₁ and EI₂ is thereby interrupted.

What is claimed is:

1. A method for detecting the state of hydration of a human or animal body, comprising:
carrying out impedance measurements on a body surface, and
carrying out and evaluating at least one bipole measurement and at least one quadrupole measurement.

2. The method according to claim 1, wherein a tissue impedance is measured with the aid of the quadrupole measurement, and a skin impedance is inferred from the values of the bipole measurement taking into account the measured tissue impedance.

3. The method according to claim 1, wherein the measurements are carried out using alternating current of prescribable frequencies, and wherein different frequencies are prescribed for measurements to determine one or more of a total body water content, an intracellular water content, and an extracellular water content.

4. The method according to claim 3, wherein algorithms are applied to infer one or more of the total body water content, a skin water content, the intracellular body water content, and the extracellular body water content from the one or more of measured and calculated impedances.

5. The method according to claim 4, wherein data concerning one or more of body size, body weight, sex, and age of a patient are input into the algorithms.

6. The method according to claim 1, wherein a preliminary measurement of electrode impedances is undertaken and taken into account in the evaluation.

7. The method according to claim 1, wherein a plurality of measurements are carried out to form mean values.

8. The method according to claim 1, wherein a measurement is additionally carried out to derive an electrocardiogram.

9. The method according to claim 1, wherein the detection of the state of hydration is incorporated into a telemedical system.

10. A device for detecting the state of hydration of a human or animal body by impedance measurements, comprising:
at least four electrodes configured to attach to one or more of hand surfaces and soles of the feet of a patient; and
a current source configured to apply alternating current to the electrodes, the electrodes being configured to be switched for bipole measurements and quadrupole measurements.

11. The device according to claim 10, wherein the current source is further configured with prescribable frequencies.

12. The device according to claim 10, further comprising a patient protection circuit.

13. The device according to claim 10, wherein the electrodes are one or more of (i) adhesive electrodes and (ii) integrated in one or more of grips and standing surfaces of the device.

14. The device according to claim 10, wherein the device is used in a telemedical system.

15. A computer program code configured to execute a method for detecting the state of hydration of a human or animal body when the program code is executed on an arithmetic unit or a control unit, the method comprising:
carrying out impedance measurements on a body surface, and
carrying out and evaluating at least one bipole measurement and at least one quadrupole measurement.

16. The computer program code according to claim 15, wherein a computer program product includes the computer program code, and stores the program code on a machine readable carrier.

17. The device according to claim 10, wherein the device includes at least eight electrodes configured to attach to the one or more of the hand surfaces and the soles of the feet of the patient.
